(19) United States
(12) Patent Application Publication
(10) Pub. No.: US 2020/0227164 A1
(43) Pub. Date: Jul. 16, 2020

(54) ENTITY CONDITION ANALYSIS BASED ON PRELOADED DATA

(71) Applicant: International Business Machines Corporation, Armonk, NY (US)

(72) Inventors: Michael Bender, Rye Brook, NY (US); Gregory J. Boss, Saginaw, MI (US); Jeremy R. Fox, Georgetown, TX (US); Craig M. Trim, Ventura, CA (US)

(21) Appl. No.: 16/244,574

(22) Filed: Jan. 10, 2019

(51) Int. Cl. G16H 50/20; G16H 50/30; G16H 10/60; G06Q 10/10; H04L 29/08; G06N 7/00

(52) U.S. Cl. CPC G16H 50/20; G16H 50/30; G06N 7/005; G06Q 10/1095; H04L 67/12; G16H 10/60

(57) ABSTRACT

In an example, a processor may receive a request for an analysis of a condition of a first entity. The processor may identify an interactive device mapped to an identifier of the first entity. The processor may identify a data type mapped to an identifier of the condition. The processor may determine that the interactive device may be configured to provide interactive data of the data type. The processor may retrieve the interactive data from the interactive device. The processor may compile condition data based on at least the identifier of the first entity, the identifier of the condition, and the interactive data. The processor may load the condition data to a device associated with a second entity. The loaded condition data may be an input to the analysis of the condition of the first entity, and the analysis may be performed by the second entity.

### BACKGROUND

The present application relates generally to computers, and computer applications, and more particularly to computer-implemented methods and systems relating to utilizing interactive device data in combination with electronic records management systems.

In some examples, records management systems may track, manage, and store records, such as electronic medical records, that may be used in various applications. With an increase in usage of interactive devices, such as Internet of Things (IoT) devices, the records stored in the records management systems may be combined with data collected from the IoT devices to execute different applications. Some examples of IoT devices may include, but not limited to, wearable devices, sensor devices, and/or other types of interactive devices that may be configured to collect data from one or more entities.

### SUMMARY

In some examples, a method of preloading data for an analysis is generally described. The method may include receiving, by a processor, a request for an analysis of a condition of a first entity. The request may include at least an identifier of the first entity and an identifier of the condition. The method may further include identifying, by the processor, an interactive device that is mapped to the identifier of the first entity. The method may further include identifying, by the processor, a data type that is mapped to the identifier of the condition. The method may further include determining, by the processor, that the interactive device is configured to provide interactive data of the data type. The method may further include retrieving, by the processor, the interactive data from the interactive device. The method may further include compiling, by the processor, condition data based on at least the identifier of the first entity, the identifier of the condition, and the interactive data. The method may further include loading, by the processor, the condition data to a device associated with a second entity. The loaded condition data may be an input to the analysis of the condition of the first entity, and the analysis may be performed by the second entity.

In some examples, a system effective to preload data for an analysis is generally described. The system may include a memory device and a hardware processor configured to be in communication with each other. The hardware processor may be configured to receive a request for an analysis of a condition of a first entity. The request may include at least an identifier of the first entity and an identifier of the condition. The hardware processor may be further configured to identify, from the memory device, an interactive device that is mapped to the identifier of the first entity. The hardware processor may be further configured to identify, from the memory device, a data type that is mapped to the identifier of the condition. The hardware processor may be further configured to determine that the interactive device is configured to provide interactive data of the data type. The hardware processor may be further configured to retrieve the interactive data from the interactive device. The hardware processor may be further configured to compile condition data based on at least the identifier of the first entity, the identifier of the condition, and the interactive data. The hardware processor may be further configured to load the condition data to a device associated with a second entity. The loaded condition data may be an input to the analysis of the condition of the first entity, and the analysis may be performed by the second entity.

In some examples, a computer program product of preloading data for an analysis is generally described. The computer program product may include a computer readable storage medium having program instructions embodied therewith. The program instructions may be executable by a processing element of a device to cause the device to perform one or more methods described herein.

Further features as well as the structure and operation of various embodiments are described in detail below with reference to the accompanying drawings. In the drawings, like reference numbers indicate identical or functionally similar elements.

### BRIEF DESCRIPTION OF THE DRAWINGS

FIG. 1 illustrates an example computer system, in one embodiment, that can be utilized to implement entity condition analysis based on preloaded data.

FIG. 2 illustrates the example system of FIG. 1 with additional details relating to an implementation of entity condition analysis based on preloaded data, in one embodiment.

FIG. 3 illustrates the example interface relating to an implementation of entity condition analysis based on preloaded data, in one embodiment.

FIG. 4 illustrates a flow diagram relating to a process, in one embodiment, to implement entity condition analysis based on preloaded data.

FIG. 5 illustrates a schematic of an example computer or processing system that may implement entity condition analysis based on preloaded data, in one embodiment.

FIG. 6 depicts a cloud computing environment according to an embodiment of the present invention.

FIG. 7 depicts abstraction model layers according to an embodiment of the present invention.

### DETAILED DESCRIPTION

To be described in more detail below, a system 100 in accordance with the present disclosure may provide a selective data collection feature, such as selecting particular IoT devices to collect IoT device data. The system 100 may also provide a data preload feature, such as loading the data selectively collected from the IoT devices to a service provider prior to services being provided by the service provider. In an example application, the IoT devices may be associated with a first entity that may be a patient, and the service provider may be a second entity such as a doctor. The data collected from the IoT devices of the first entity (patient) may be related to a condition (e.g., health, illness, symptom, and/or other conditions) of the first entity. The data collected from the IoT devices of the first entity may be loaded to devices being operated by the second entity (doctor). In an example, the collected data may be loaded to the devices prior to an appointment or meeting between the first and second entity. Thus, at an occurrence of the appointment, the devices being operated by the second entity may be preloaded with the collected data, where the preloaded data may be used by the second entity. For example, the preloaded data may be used by the second entity as an input to an analysis (e.g., treatment) of the condition of the first entity. Further, the system 100 may select the data to be collected from the IoT devices by using a cognitive approach to forecast the types of data that may be used by the second entity in the analysis of the condition of the first entity. At least a portion of the system 100 may be implemented as a standalone system or application, or may be implemented as an add-on to another system such as a records management system, an autonomous appointment scheduling system, a service provider identification and location system, and/or other types of systems. Thus, the system 100 adds the data preload feature to another system that may lack the data preload feature. The system with the added data preload feature may be used by the second entity to enhance an efficiency in an analysis of the condition due to a reduction in time to collect data during an appointment or meeting between the first entity and the second entity. Further, the system 100 adds a feature of selective data collection from IoT devices to another system that may lack the selective data collection feature. The system with the added selective data collection feature may output the types of IoT device data that may be needed from the second entity to analyze the condition of the first entity. Thus, the system with the added selective data collection feature may present the first entity with information about different service providers such that the first entity may use the presented information to select a service provider to analyze the condition.

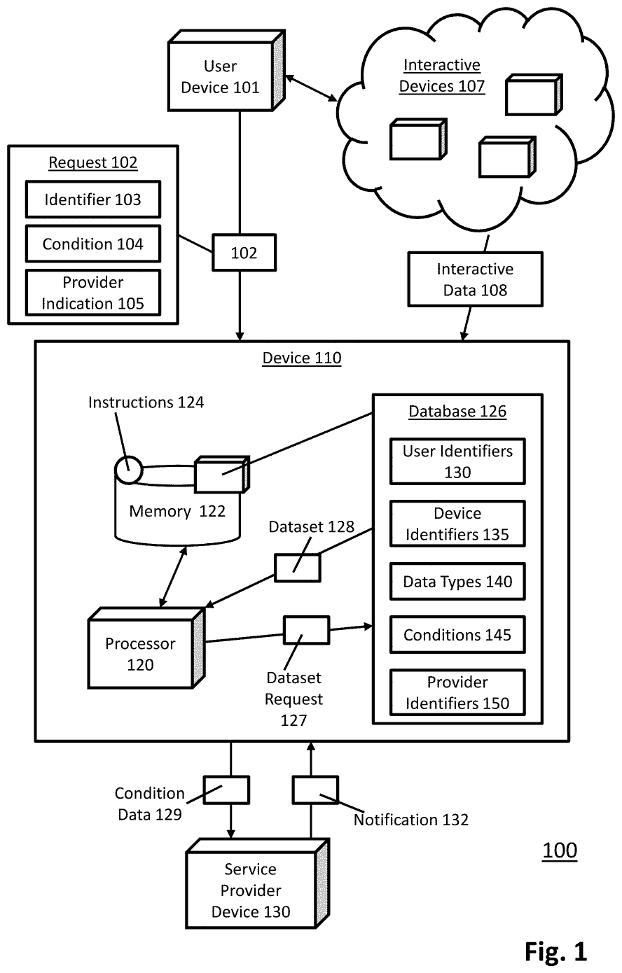

FIG. 1 illustrates an example computer system, in one embodiment, that can be utilized to implement entity condition analysis based on preloaded data, arranged in accordance with at least some embodiments described herein. In some examples, the system 100 may be implemented in a computer device 110 (“device 110”). The device 110 may include a processor 120 and a memory 122 configured to be in communication with each other. In some examples, the processor 120 may be a central processing unit of the device 110, and may be configured to control operations of the memory 122 and/or other components of the device 110. In some examples, the system 100 and/or the device 110 may include additional hardware components, such as programmable logic devices, microcontrollers, memory devices, and/or other hardware components, that may be configured to perform respective tasks of the methods described in the present disclosure. In some examples, the processor 120 may be configured to execute software modules that include instructions to perform each respective task of the methods described in the present disclosure. In some examples, the processor 120 and the memory 122 may be components of a cloud computing platform that may be employed to implement the methods described in accordance with the present disclosure.

The memory 122 is configured to selectively store instructions executable by the processor 120. For example, in one embodiment, the memory 122 may store a set of instructions 124 (“instructions 124”), where the instructions 124 include instructions, such as executable code, related to machine learning algorithms, database algorithms, and/or other algorithms or techniques, which may implement the system 100. The processor 120 is configured to execute one or more portions of the instructions 124 in order to facilitate implementation of the system 100. In some examples, the instructions 124 may be packaged as a standalone application that may be installed on the device 110, such that the instructions 124 may be executed by the processor 120 to implement the system 100. In some examples, the instructions 124 may be stored in a programmable hardware component that may be embedded as part of the processor 120.

The memory 122 may further store a database 126, where the database 126 includes data that may be queried by the processor 120 (further described below). In an example, the database 126 may store one or more tables that include user identifiers 130, one or more device identifiers 135, one or more data types 140, one or more conditions 145, and one or more service provider identifiers 150 (“provider identifiers 150”). The user identifiers 130 may be identifiers of one or more entities, such as users (or patients) of the system 100. The device identifiers 135 may be identifiers effective to identify one or more interactive devices 107, such as a brand, model number, firmware version, and/or other attributes, of interactive devices 107. The interactive devices 107, in some examples, may include computer devices, wearable devices, sensors, and/or other types of devices. The data types 140 may be types of data that may be collected by one or more of interactive devices 107, and each data type may relate to one or more conditions. For example, the data types 140 may include sleep pattern, diet pattern, exercise pattern, glucose level, heart rate, blood pressure, body temperature, height, weight, various types of biometric data, and/or other data types. The conditions 145 may include one or more health related conditions and/or symptoms, such as chronic tiredness, insomnia, weight loss, weight gain, fever, and/or other health related conditions. The provider identifiers 150 may uniquely identify one or more service providers such as health professionals, consultants, doctors, and/or other types of providers. The database 126 may further store relationships and mappings between one or more of the identifiers 130, the device identifiers 135, the data types 140, the conditions 145, and the provider identifiers 150 (further described below). In some examples, the database 126 may be stored in a memory device different from memory 122, such as being stored at a memory device in a cloud computing platform. The data stored in the database 126 may be data that are collected by the system 100 based on authorizations by one or more users of the system 100. The embodiments of the present invention provide features that allow the users of the system 100 to select whether to participate in data collection that may be performed by the system 100. For example, the system 100 may request authorization from the user device 101 to collect data such as an identifier of the first entity and to collect data from the interactive devices 107. In some examples, the system 100 may terminate data collection from the user device 101 and/or the interactive devices 107 in response to receiving an opt-out notification from the first entity. In some examples, the system 100 may push a notification to the user device 101 and/or the interactive devices 107 when the system 100 begins to perform any data collection, tracking, and/or monitoring relating to the first entity.

In an example, a user device 101 may send a request 102 to device 110. The user device 101 may be associated with, or operated by, a first entity (e.g., a user of system 100, a patient, or other types of entity). The user device 101 may be, for example, a desktop computer, a laptop computer, a tablet device, a cellular phone, a wearable device, and/or other types of electronic devices. The request 102 may be a request for an analysis of a condition 104 of the first entity associated with the user device 101. For example, the request 102 may be a request to analyze a condition of chronic tiredness being experienced by the first entity. In some examples, the request 102 may include the condition 104, an identifier 103 of the first entity, and/or a provider indication 105. The identifier 103 of the first entity may be, for example, a username, a phone number, an identification number, an email address, a name (first, middle, and/or last name), and/or other types of identifiers effective to uniquely identify the first entity. The provider indication 105 may be a name of the service provider, a type and/or an attribute of a service provider such as a particular medical specialty, a geographical location (address, city, town, state, zip code and/or other location information), and/or other information effective to identify one or more service providers. In some examples, the contents of the request 102 (e.g., the identifier 103, the condition 104, and/or the provider indication 105) may be structured data in a machine-readable format.

The device 110 may receive the request 102 and may extract the identifier 103, the condition 104, and/or the provider indication 105 from the request 102. The identifier 103 may be among the user identifiers 130 stored in the database 126, the condition 104 may be among the conditions 145 stored in the database 126, and a provider indication 105 may be among the provider identifiers 150 stored in the database 126.

In an example embodiment, the processor 120 may generate a dataset request 127 to query the database 126 to return a dataset 128. For example, the dataset request 127 may include the identifier 103, the condition 104, and/or the provider indication 105 extracted from the request 102. In an example, the dataset request 127 may request identifiers of one or more interactive devices 107, which may be among the device identifiers 135 in the database 126, that are mapped to the identifier 103. In another example, the dataset request 127 may request one or more data types, among data types 140, that are mapped to condition 104. In another example, the dataset request 127 may request one or more provider identifiers, among the provider identifiers 150, that satisfies or complies with the provider indication 105.

The dataset 128 returned from the database 126 may include identifications of one or more interactive devices 107 mapped to the identifier 103, one or more data types mapped to the condition 104, and/or one or more provider identifiers that satisfied the provider indication 105. The processor 120 may retrieve interactive data 108 from interactive devices 107 identified in the dataset 128, where the interactive data 108 may be of the data type identified in the dataset 128. The interactive data 108 may include data that corresponds to the condition 104 indicated by the request 102. For example, one of the interactive devices 107 may be a wearable device configured to track sleep pattern of the first entity, where sleep pattern may be a data type mapped to a condition of chronic tiredness in database 126.

The processor 120 may generate or compile condition data 129 based on the identifier 103, the condition 104, the dataset 128, and/or the interactive data 108. In some examples, the condition data 129 may include image data that may be rendered by the processor 120 to display a report in a user interface (further described below). The displayed report may include one or more of the identifier 103, the condition 104, the dataset 128, and/or the interactive data 108. The processor 120 may load the condition data 129 to a service provider device 130 associated with, or operated by, a second entity such as a service provider.

In an example, the service provider device 130 may send a notification 132 to device 110, where the notification 132 may indicate whether the condition data 129 loaded into the service provider device 130 is sufficient. If the notification 132 indicates that the loaded condition data 129 is insufficient, processor 120 may, autonomously, retrieve additional interactive data 108 from interactive devices 107. For example, the loaded condition data 129 may include a one week span of sleep pattern of the first entity. The notification 132 may indicate that one week is insufficient, and request a two week span of sleep pattern of the first entity. The processor 120 may retrieve an additional week of sleep pattern tracked by one or more of interactive devices 107. The processor 120 may re-compile the condition data 129 using the additionally retrieved interactive data, and may reload the updated condition data 129 to the service provider device 130. The service provider 130 may continue to update and send notification 132 indicating whether the loaded condition data 129 is sufficient, and the processor 120 may continue to retrieve interactive data 108 from interactive devices 107 until the notification 132 indicates that the loaded condition data 129 is sufficient.

In some examples, the provider indication 105 may be an attribute of a service provider, such as a geographical location. The processor 120 may query the database 126 to identify one or more provider identifiers of service providers that are located within a threshold distance from a location of the first entity. The query may return a dataset corresponding to each identified service provider, where each dataset may indicate or include different interactive devices 107 and/or data types. For example, each identified service provider may be a candidate entity that uses a respective set of data types to analyze the condition 104 in the request 102. Thus, each dataset may include a respective set of data types, and may include a respective set of interactive devices that are configured to provide the respective set of data types. The processor 120, based on the data types and interactive devices indicated by the dataset 128, may selectively retrieve interactive data 108 from the interactive devices 107. For example, if a dataset indicates sleep pattern and exercise pattern, which are mapped to a particular wearable device in the database 126, the processor 120 may retrieve sleep pattern and exercise pattern data from the particular wearable device. The processor 120 may send the identified service providers and corresponding dataset 128 to the user device 101 to prompt the first entity to select one or more service providers. For example, the processor 120 may present to the first entity a first service provider that may recommend changing sleep pattern to treat chronic tiredness, and may present a second service provider that may recommend changing exercise patterns to treat chronic tiredness. The first entity may use the user device 101 to make a selection of service provider, and may send an indication to the device 110 to indicate the selection. The processor 120 may receive the selection from the user device 101 and may autonomously arrange or schedule an appointment with the service provider indicated in the selection.

In some examples, the provider indication 105 included in the request 102 may include a constraint, or criteria, of a service provider, such as a criteria indicating an experience level (e.g., a minimum number of years) of the second entity. The processor 120 may query the database 126 to identify one or more service providers that satisfies the criteria. The query may return a dataset 128 for each identified service provider, where each dataset 128 may indicate or include different interactive devices 107 and/or data types. The processor 120 may apply machine learning techniques on the dataset 128 to train a model that indicates probability of data types that may be used by the service providers satisfying the criteria. For example, the model may indicate an 85% probability that a service provider with X years of experience will analyze sleep patterns, and a 65% probability that the service provider will analyze exercise patterns, when treating chronic tiredness. The processor 120 may output results from the model to the user device 101 in order for the first entity to view the different probabilities of data types and to select a service provider.

In some examples, the processor 120 may use the model to autonomously retrieve interactive data 108 from interactive devices 107. For example, the provider indication 105 may be an identifier of a particular service provider. The processor 120 may, based on the database 126, determine that the particular service provider has X years of experience. The processor 120 may use the model trained based on datasets of service providers with X years of experience to forecast the data types that may be used by the particular service provider in analyzing condition 104. The forecasted data types that may be data types that correspond to a probability above 60% indicated in the trained model. The processor 120 may be configured to retrieve interactive data of the forecasted data types from the interactive devices 107. Thus, using the example described above, the processor 120 may autonomously retrieve sleep pattern data and exercise pattern data from one or more interactive devices 107 that are configured to track sleep and exercise patterns.

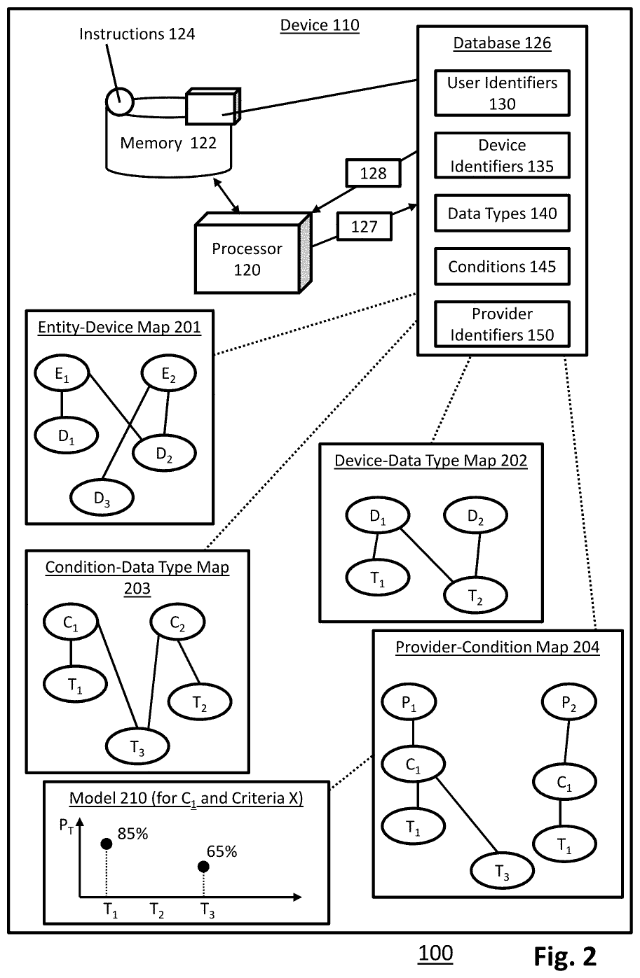

FIG. 2 illustrates the example system of FIG. 1 with additional details relating to an implementation of entity condition analysis based on preloaded data, in one embodiment. FIG. 2 may include components that are labeled identically to components of FIG. 1, which will not be described again for the purposes of clarity. The description of FIG. 2 may reference at least some of the components of FIG. 1.

The database 126, in some examples, may be a relational database that relates to a plurality of tables. In an example, the database 126 may store relationships and mappings between one or more of the identifiers 130, the device identifiers 135, the data types 140, the conditions 145, and the provider identifiers 150. In an example shown in FIG. 2, the database 126 may store an entity-device map 201, a device-data type may 202, a condition-data type map 203, and a provider-condition map 204, herein map 201, map 202, map 203, map 204, respectively.

Map 201 may map user identifiers 130 to device identifiers 135. In an example shown in FIG. 2, a first user may be identified by an user identifier E₁, and E₁ may be mapped to device identifiers D₁ and D₂. The mapping between E₁ and D₁, D₂, indicates that the first user has registered interactive devices identified by D₁ and D₂ in the system 100, which indicates that the first user is in possession of interactive devices D₁ and D₂. Similarly, a second user may be identified by a user identifier E₂, and E₂ may be mapped to device identifiers D₂ and D₃. The mapping between E₂ and D₂, D₃, indicates that the first user has registered interactive devices identified by D₂ and D₃ in the system 100, which indicates that the second user is in possession of interactive devices D₂ and D₃.

Map 202 may map device identifiers 135 to data types 140. In an example shown in FIG. 2, D₁ may be mapped to data types T₁ and T₂, and D₂ may be mapped to data type T₂. The mapping between D₁ and T₁, T₂, indicates that the interactive device D₁ is configured to collect data of the data types T₁, T₂. The mapping between D₂ and T₂, indicates that the interactive device D₂ is configured to collect data of the data type T₂. For example, interactive device D₁ may be a smart watch configured to collect sleep pattern (T₁) and exercise pattern (T₂).

Map 203 may map conditions 145 to data types 140. In an example shown in FIG. 2, a condition C₁ may be mapped to data types T₁ and T₃, and a condition C₂ may be mapped to data types T₂ and T₃. The mapping between C₁ and T₁, T₃, indicates that the condition C₁ may be analyzed by a service provider using data of data types T₁, T₃. Similarly, the mapping between C₂ and T₂, T₃, indicates that the condition C₂ may be analyzed by a service provider using data of data types T₂, T₃. For example, condition C₁ may be chronic tiredness, and data types of sleep pattern (T₁) and exercise pattern (T₂) may have been used by one or more service providers in analyzing the condition C₁.

Map 204 may include more than one level of mapping, such as a first map level that relates provider identifiers 150 to conditions 145, and a second map level that relates conditions 145 to data types 140 based on the provider. In an example shown in FIG. 2, a provider P₁ may be mapped to condition C₁, and condition C₁ may be mapped to data types T₁ and T₃. The mappings linked to provider P₁ indicates that provider P₁ has treated or analyzed condition C₁ using data types T₁ and T₃. Similarly, a provider P₂ may be mapped to condition C₁, and condition C₁ may be mapped to data type T₁. The mappings linked to provider P₂ may indicate that provider P₂ has treated or analyzed condition C₂ using data type T₁.

Using the maps among the database 126, the processor 120 may query the database 126 to determine dataset 128, and selectively retrieve interactive data 108 from interactive devices 107. In an example, the request 102 may be a request to identify a second entity, or a service provider, that may analyze a condition of chronic tiredness, labeled as condition C₁, being experienced by an entity E₁. Processor 120 may generate the dataset request 127 to query the database 126 to return a dataset 128. The dataset request 127 may request one or more data types mapped to condition C₁, and may request one or more interactive devices mapped to E₁. Using map 201, the processor 120 may identify interactive devices D₁ and D₂ mapped to E₁. Using map 203, the processor 120 may identify data types T₁ and T₃ mapped to C₁. The processor 120 may determine whether the identified interactive devices D₁ and D₂ are configured to provide data of the identified data types T₁ and T₃. Using map 202, the processor 120 may determine that device D₁ may be configured to collect and provide data of data type T₁, and may determine that device D₂ may not be configured to collect and provide data of data types T₁ and T₃.

Processor 120 may generate dataset 128 including a mapping between device D₁ and data type T₁. In examples where the database 126 is outside of the device 110, such as being stored in a memory device on a cloud computing platform, the dataset 128 may be generated by a server or processor of the cloud computing platform and transmitted to the device 110. The processor 120, based on the mapping between device D₁ and data type T₁ in the dataset 128, may send a request to device D₁ to request a retrieval of interactive data 108 of the data type T₁. The processor 120 may retrieve the interactive data 108 of the data type T₁ from interactive device D₁. The processor 120 may compile condition data 129, such as by aggregating identifier E₁, interactive device D₁, the retrieved interactive data 108, to generate a report, or to generate an image data that may be rendered into a user interface to be displayed by the service provider device 130 and/or user device 101. The processor 120 may further compile the condition data 129 to indicate that data of data type T₃ is not available (C₁ is mapped to T₃, but E₁ is not mapped to a device that can provide data of T₃).

In an example shown in FIG. 2, processor 120 may train a model 210 using data stored in the database 126. For example, provider P₁ and P₂ may both satisfy a criteria X, such as having a certain number of years of experience. Using map 204, processor 120 may train a model that indicates a probability of usage P_{T}, where each P_{T} indicates a probability that a provider satisfying criteria X will use data type T in an analysis of condition C₁. In an example, the map 204 may indicate that 85% of service providers have used data type T₁ in treating and analyzing condition C₁, 0% of service providers have used data type T₂ in treating and analyzing condition C₁, and 65% of service providers have used data type T₃ in treating and analyzing condition C₁. Thus, processor 120 may train the model 210 to indicate that there is a 85% probability that a provider satisfying criteria X may need data of data type T₁ to analyze condition C₁, data of data type T₂ may not be needed, and there is a 85% probability that a provider satisfying criteria X may need data of data type T₁ to analyze condition C₁.

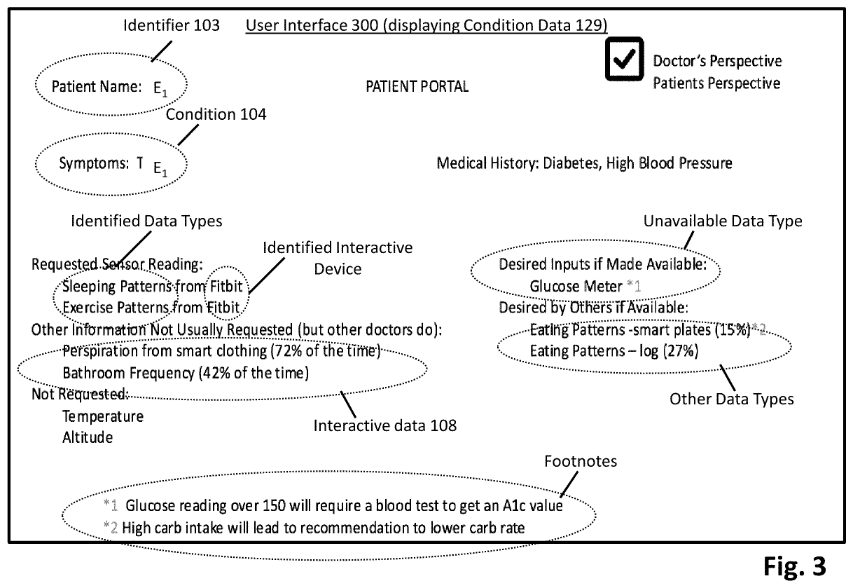

FIG. 3 illustrates the example interface relating to an implementation of entity condition analysis based on preloaded data, in one embodiment. FIG. 3 may include components that are labeled identically to components of FIGS. 1-2, which will not be described again for the purposes of clarity. The description of FIG. 3 may reference at least some of the components of FIGS. 1-2.

In an example shown in FIG. 3, a report that may be a part of condition data 129 may be displayed in a user interface 300, where the user interface 300 may be displayed by the user device 101 and/or the service provider device 130. Access to the report may be restricted, for example, so that only service providers approved by the first entity can access the report, in order to protect the identity and information of the first entity from unauthorized entities. The report may include the identifier 103 of the first entity, the condition 104, the identified data types, the identified interactive devices, the retrieved interactive data 108, and/or other information. For example, the report may further include medical history of the first entity, where the medical history may be stored as historical data in the memory 122 and/or the database 126. The report may further indicate data types that were not requested to be retrieved, such as temperature (e.g., due to having a probability of usage below a threshold indicated by, for example, model 210 described above). The report may further indicate data types that were not available for retrieval (e.g., T₃ described above), labeled as unavailable data type in FIG. 3. The report may further indicate “other data types”, which may be data types requested by service providers other than service providers associated with the provider indication 105 in the request 102. For example, if provider indication 105 indicates criteria X, the report may indicate data types that have been requested by service providers that do not satisfy criteria X in analysis of the condition 104.

The report may further include one or more footnotes that may indicate recommendations for the first entity or the second entity, where the recommendations may relate to utilization of the interactive data 108 collected, course of actions to be perform by the first entity and/or the second entity prior to the second entity analyzing the condition 104 of the first entity, and/or other recommendations. For example, the footnote shown in FIG. 3 indicates that glucose reading over 150 may require a blood test, and thus, the second entity may arrange a blood test for the first entity prior to an appointment between the first entity and the second entity if a glucose reading of the first entity is over 150.

By indicating data types that may not be available from the first entity, the condition data 129 provides suggestions to the first entity for obtaining particular interactive devices that may be beneficial to the first entity (e.g., trigger the first entity to obtain a glucose meter). Also, by indicating data types that may be used by other service providers, a service provider viewing the condition data 129 on the user interface 300 may become aware of a potential to use new data types in further analysis of the condition 104.

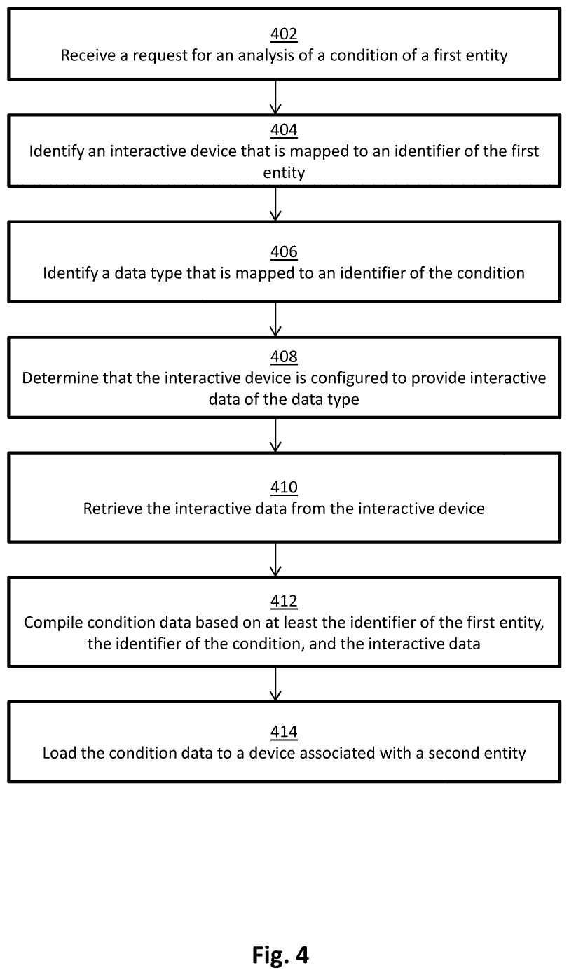

FIG. 4 illustrates a flow diagram relating to a process, in one embodiment, to implement entity condition analysis based on preloaded data, arranged in accordance with at least some embodiments presented herein. The process in FIG. 4 may be implemented using, for example, computer system 100 discussed above. An example process may include one or more operations, actions, or functions as illustrated by one or more of blocks 402, 404, 406, 408, 410, 412, and/or 414. Although illustrated as discrete blocks, various blocks may be divided into additional blocks, combined into fewer blocks, eliminated, or performed in parallel, depending on the desired implementation.

Processing may begin at block 402, where a processor receives a request for an analysis of a condition of a first entity. The request includes at least an identifier of the first entity and an identifier of the condition. Processing may continue from block 402 to block 404. At block 404, the processor identifies an interactive device that is mapped to an identifier of the first entity.

Processing may continue from block 404 to block 406. At block 406, the processor identifies a data type that is mapped to an identifier of the condition. Processing may continue from block 406 to block 408. At block 408, the processor determines that the interactive device is configured to provide interactive data of the data type. Processing may continue from block 408 to block 410. At block 410, the processor retrieves the interactive data from the interactive device.

Processing may continue from block 410 to block 412. At block 412, the processor compiles condition data based on at least the identifier of the first entity, the identifier of the condition, and the interactive data. Processing may continue from block 412 to block 414. At block 414, the processor loads the condition data to a device associated with a second entity. The loaded condition data is an input to the analysis of the condition of the first entity, and the analysis is to be performed by the second entity.

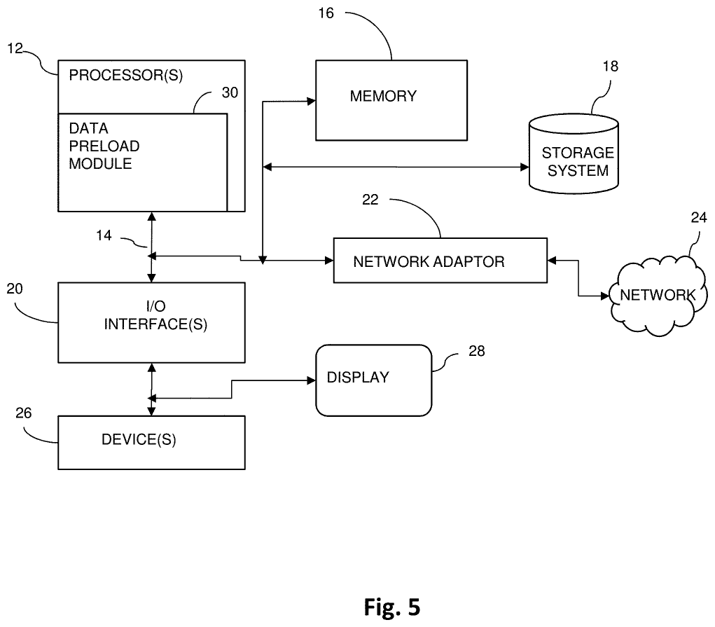

FIG. 5 illustrates a schematic of an example computer or processing system that may implement entity condition analysis based on preloaded data in one embodiment of the present disclosure. The computer system is only one example of a suitable processing system and is not intended to suggest any limitation as to the scope of use or functionality of embodiments of the methodology described herein. The processing system shown may be operational with numerous other general purpose or special purpose computing system environments or configurations. Examples of well-known computing systems, environments, and/or configurations that may be suitable for use with the processing system shown in FIG. 5 may include, but are not limited to, personal computer systems, server computer systems, thin clients, thick clients, handheld or laptop devices, multiprocessor systems, microprocessor-based systems, set top boxes, programmable consumer electronics, network PCs, minicomputer systems, mainframe computer systems, supercomputers, and distributed cloud computing environments that include any of the above systems or devices, and the like.

The computer system may be described in the general context of computer system executable instructions, such as program modules, being executed by a computer system. Generally, program modules may include routines, programs, objects, components, logic, data structures, and so on that perform particular tasks or implement particular abstract data types. The computer system may be practiced in distributed cloud computing environments where tasks are performed by remote processing devices that are linked through a communications network. In a distributed cloud computing environment, program modules may be located in both local and remote computer system storage media including memory storage devices.

The components of computer system may include, but are not limited to, one or more processors or processing units 12, a system memory 16, and a bus 14 that couples various system components including system memory 16 to processor 12. The processor 12 may include a module 30 (e.g., data preload module 30) that performs the methods described herein. The module 30 may be programmed into the integrated circuits of the processor 12, or loaded from memory 16, storage device 18, or network 24 or combinations thereof.

Bus 14 may represent one or more of any of several types of bus structures, including a memory bus or memory controller, a peripheral bus, an accelerated graphics port, and a processor or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnects (PCI) bus.

Computer system may include a variety of computer system readable media. Such media may be any available media that is accessible by computer system, and it may include both volatile and non-volatile media, removable and non-removable media.

System memory 16 can include computer system readable media in the form of volatile memory, such as random access memory (RAM) and/or cache memory or others. Computer system may further include other removable/non-removable, volatile/non-volatile computer system storage media. By way of example only, storage system 18 can be provided for reading from and writing to a non-removable, non-volatile magnetic media (e.g., a “hard drive”). Although not shown, a magnetic disk drive for reading from and writing to a removable, non-volatile magnetic disk (e.g., a “floppy disk”), and an optical disk drive for reading from or writing to a removable, non-volatile optical disk such as a CD-ROM, DVD-ROM or other optical media can be provided. In such instances, each can be connected to bus 14 by one or more data media interfaces.

Computer system may also communicate with one or more external devices 26 such as a keyboard, a pointing device, a display 28, etc.; one or more devices that enable a user to interact with computer system; and/or any devices (e.g., network card, modem, etc.) that enable computer system to communicate with one or more other computing devices. Such communication can occur via Input/Output (I/O) interfaces 20.

Still yet, computer system can communicate with one or more networks 24 such as a local area network (LAN), a general wide area network (WAN), and/or a public network (e.g., the Internet) via network adapter 22. As depicted, network adapter 22 communicates with the other components of computer system via bus 14. It should be understood that although not shown, other hardware and/or software components could be used in conjunction with computer system. Examples include, but are not limited to: microcode, device drivers, redundant processing units, external disk drive arrays, RAID systems, tape drives, and data archival storage systems, etc.

The present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

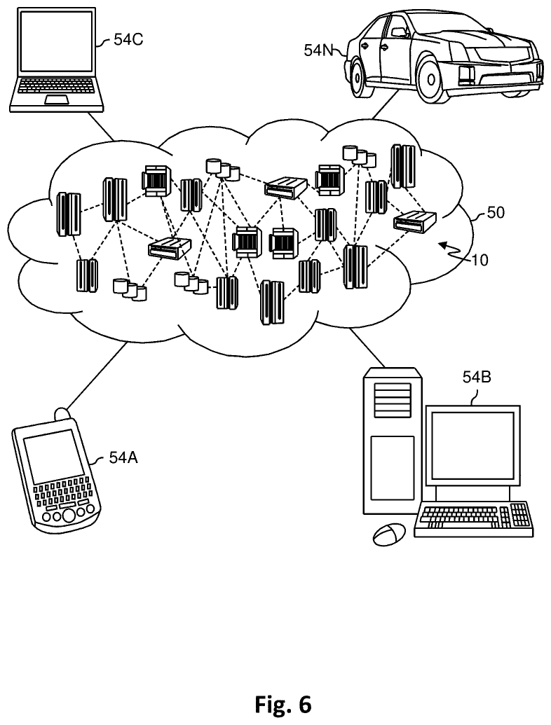

FIG. 6 depicts a cloud computing environment according to an embodiment of the present invention. It is to be understood that although this disclosure includes a detailed description on cloud computing, implementation of the teachings recited herein are not limited to a cloud computing environment. Rather, embodiments of the present invention are capable of being implemented in conjunction with any other type of computing environment now known or later developed.

Cloud computing is a model of service delivery for enabling convenient, on-demand network access to a shared pool of configurable computing resources (e.g., networks, network bandwidth, servers, processing, memory, storage, applications, virtual machines, and services) that can be rapidly provisioned and released with minimal management effort or interaction with a provider of the service. This cloud model may include at least five characteristics, at least three service models, and at least four deployment models.

Characteristics are as follows:

On-demand self-service: a cloud consumer can unilaterally provision computing capabilities, such as server time and network storage, as needed automatically without requiring human interaction with the service's provider.

Broad network access: capabilities are available over a network and accessed through standard mechanisms that promote use by heterogeneous thin or thick client platforms (e.g., mobile phones, laptops, and PDAs).

Resource pooling: the provider's computing resources are pooled to serve multiple consumers using a multi-tenant model, with different physical and virtual resources dynamically assigned and reassigned according to demand. There is a sense of location independence in that the consumer generally has no control or knowledge over the exact location of the provided resources but may be able to specify location at a higher level of abstraction (e.g., country, state, or datacenter).

Rapid elasticity: capabilities can be rapidly and elastically provisioned, in some cases automatically, to quickly scale out and rapidly released to quickly scale in. To the consumer, the capabilities available for provisioning often appear to be unlimited and can be purchased in any quantity at any time.

Measured service: cloud systems automatically control and optimize resource use by leveraging a metering capability at some level of abstraction appropriate to the type of service (e.g., storage, processing, bandwidth, and active user accounts). Resource usage can be monitored, controlled, and reported, providing transparency for both the provider and consumer of the utilized service.

Service Models are as follows:

Software as a Service (SaaS): the capability provided to the consumer is to use the provider's applications running on a cloud infrastructure. The applications are accessible from various client devices through a thin client interface such as a web browser (e.g., web-based e-mail). The consumer does not manage or control the underlying cloud infrastructure including network, servers, operating systems, storage, or even individual application capabilities, with the possible exception of limited user-specific application configuration settings.

Platform as a Service (PaaS): the capability provided to the consumer is to deploy onto the cloud infrastructure consumer-created or acquired applications created using programming languages and tools supported by the provider. The consumer does not manage or control the underlying cloud infrastructure including networks, servers, operating systems, or storage, but has control over the deployed applications and possibly application hosting environment configurations.

Infrastructure as a Service (IaaS): the capability provided to the consumer is to provision processing, storage, networks, and other fundamental computing resources where the consumer is able to deploy and run arbitrary software, which can include operating systems and applications. The consumer does not manage or control the underlying cloud infrastructure but has control over operating systems, storage, deployed applications, and possibly limited control of select networking components (e.g., host firewalls).

Deployment Models are as follows:

Private cloud: the cloud infrastructure is operated solely for an organization. It may be managed by the organization or a third party and may exist on-premises or off-premises.

Community cloud: the cloud infrastructure is shared by several organizations and supports a specific community that has shared concerns (e.g., mission, security requirements, policy, and compliance considerations). It may be managed by the organizations or a third party and may exist on-premises or off-premises.

Public cloud: the cloud infrastructure is made available to the general public or a large industry group and is owned by an organization selling cloud services.

Hybrid cloud: the cloud infrastructure is a composition of two or more clouds (private, community, or public) that remain unique entities but are bound together by standardized or proprietary technology that enables data and application portability (e.g., cloud bursting for load-balancing between clouds).

A cloud computing environment is service oriented with a focus on statelessness, low coupling, modularity, and semantic interoperability. At the heart of cloud computing is an infrastructure that includes a network of interconnected nodes.

Referring now to FIG. 6, illustrative cloud computing environment 50 is depicted. As shown, cloud computing environment 50 includes one or more cloud computing nodes 10 with which local computing devices used by cloud consumers, such as, for example, personal digital assistant (PDA) or cellular telephone 54A, desktop computer 54B, laptop computer 54C, and/or automobile computer system 54N may communicate. Nodes 10 may communicate with one another. They may be grouped (not shown) physically or virtually, in one or more networks, such as Private, Community, Public, or Hybrid clouds as described hereinabove, or a combination thereof. This allows cloud computing environment 50 to offer infrastructure, platforms and/or software as services for which a cloud consumer does not need to maintain resources on a local computing device. It is understood that the types of computing devices 54A-N shown in FIG. 6 are intended to be illustrative only and that computing nodes 10 and cloud computing environment 50 can communicate with any type of computerized device over any type of network and/or network addressable connection (e.g., using a web browser).

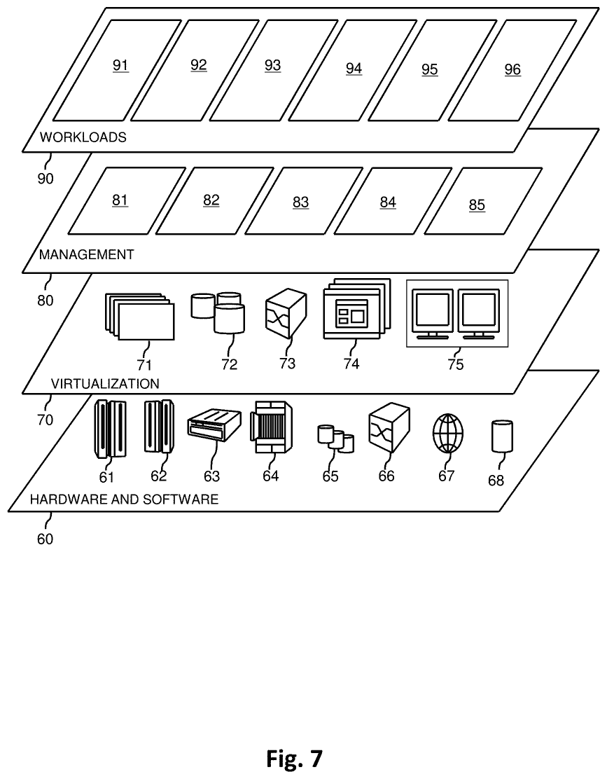

FIG. 7 depicts abstraction model layers according to an embodiment of the present invention. Referring now to FIG. 7 a set of functional abstraction layers provided by cloud computing environment 50 (FIG. 6) is shown. It should be understood in advance that the components, layers, and functions shown in FIG. 8 are intended to be illustrative only and embodiments of the invention are not limited thereto. As depicted, the following layers and corresponding functions are provided:

Hardware and software layer 60 includes hardware and software components. Examples of hardware components include: mainframes 61; RISC (Reduced Instruction Set Computer) architecture based servers 62; servers 63; blade servers 64; storage devices 65; and networks and networking components 66. In some embodiments, software components include network application server software 67 and database software 68.

Virtualization layer 70 provides an abstraction layer from which the following examples of virtual entities may be provided: virtual servers 71; virtual storage 72; virtual networks 73, including virtual private networks; virtual applications and operating systems 74; and virtual clients 75.

In one example, management layer 80 may provide the functions described below. Resource provisioning 81 provides dynamic procurement of computing resources and other resources that are utilized to perform tasks within the cloud computing environment. Metering and Pricing 82 provide cost tracking as resources are utilized within the cloud computing environment, and billing or invoicing for consumption of these resources. In one example, these resources may include application software licenses. Security provides identity verification for cloud consumers and tasks, as well as protection for data and other resources. User portal 83 provides access to the cloud computing environment for consumers and system administrators. Service level management 84 provides cloud computing resource allocation and management such that required service levels are met. Service Level Agreement (SLA) planning and fulfillment 85 provide pre-arrangement for, and procurement of, cloud computing resources for which a future requirement is anticipated in accordance with an SLA.

Workloads layer 90 provides examples of functionality for which the cloud computing environment may be utilized. Examples of workloads and functions which may be provided from this layer include: mapping and navigation 91; software development and lifecycle management 92; virtual classroom education delivery 93; data analytics processing 94; transaction processing 95; and condition analysis with preloaded data 96.

The corresponding structures, materials, acts, and equivalents of all means or step plus function elements, if any, in the claims below are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

What is claimed is:

1. A computer-implemented method comprising:
receiving, by a processor, a request for an analysis of a condition of a first entity, wherein the request comprises at least an identifier of the first entity and an identifier of the condition;
identifying, by the processor, an interactive device that is mapped to the identifier of the first entity;
identifying, by the processor, a data type that is mapped to the identifier of the condition;
determining, by the processor, that the interactive device is configured to provide interactive data of the data type;
retrieving, by the processor, the interactive data from the interactive device;
compiling, by the processor, condition data based on at least the identifier of the first entity, the identifier of the condition, and the interactive data; and
loading, by the processor, the condition data to a device associated with a second entity, wherein the loaded condition data is an input to the analysis of the condition of the first entity, and the analysis is to be performed by the second entity.

2. The computer-implemented method of claim 1, further comprising scheduling, autonomously by the processor, an appointment with the second entity to analyze the condition of the first entity.

3. The computer-implemented method of claim 1, further comprising:
receiving, by the processor, a notification from the device indicating an insufficiency of the loaded condition data;
retrieving, by the processor, additional interactive data of the data type from the interactive device;
compiling, by the processor, new condition data based on the additional interactive data; and
loading, by the processor, the new condition data to the device.

4. The computer-implemented method of claim 1, wherein the request indicates an identifier of the second entity, and the method comprising identifying the interactive device and the data type based on the second entity.

5. The computer-implemented method of claim 1, wherein the request indicates an attribute, and the method comprising:
identifying, by the processor, a candidate second entity with the attribute;
identifying, by the processor, at least one data type mapped to the candidate second entity;
sending, by the processor, an identifier of the candidate second entity and the at least one data type to a device of the first entity; and
receiving, by the processor, an indication that the candidate second entity is selected as the second entity to analyze the condition of the first entity.

6. The computer-implemented method of claim 1, wherein the request indicates a criteria, and the method comprising:
identifying, by the processor, a candidate second entity that satisfies with the criteria;
identifying, by the processor, at least one data type mapped to the candidate second entity;
training, by the processor, a model based on the at least one data type, wherein the model indicates a probability of usage of each data type among the at least one data type by the candidate second entity;
sending, by the processor, the probabilities of usage of each data type to a device of the first entity; and
receiving, by the processor, a selection indicating that the candidate second entity is selected as the second entity to analyze the condition of the first entity.

7. The computer-implemented method of claim 6, wherein the request includes an identifier of the second entity, and the method further comprising retrieving, by the processor, the interactive data from the interactive device based on the probabilities of usage of the data type by the second entity.

8. A system comprising:
a memory device;
a hardware processor configured to be in communication with the memory device, the hardware processor being configured to:
receive a request for an analysis of a condition of a first entity, wherein the request comprises at least an identifier of the first entity and an identifier of the condition;
identify, from the memory device, an interactive device that is mapped to the identifier of the first entity;
identify, from the memory device, a data type that is mapped to the identifier of the condition;
determine that the interactive device is configured to provide interactive data of the data type;
retrieve the interactive data from the interactive device;
compile condition data based on at least the identifier of the first entity, the identifier of the condition, and the interactive data; and
load the condition data to a device associated with a second entity, wherein the loaded condition data is an input to the analysis of the condition of the first entity, and the analysis is to be performed by the second entity.

9. The system of claim 8, wherein the hardware processor is further configured to schedule, autonomously, an appointment with the second entity to analyze the condition of the first entity.

10. The system of claim 8, wherein the hardware processor is further configured to:
receive a notification from the device indicating an insufficiency of the loaded condition data;
retrieve additional interactive data of the data type from the interactive device;
compile new condition data based on the additional interactive data; and
load the new condition data to the device.

11. The system of claim 8, wherein the request indicates an identifier of the second entity, and the hardware processor is further configured to identify the interactive device and the data type based on the second entity.

12. The system of claim 8, wherein the request indicates an attribute, and the hardware processor is further configured to:
identify a candidate second entity with the attribute;
identify at least one data type mapped to the candidate second entity;
send an identifier of the candidate second entity and the at least one data type to a device of the first entity; and
receive a selection indicating that the candidate second entity is selected as the second entity to analyze the condition of the first entity.

13. The system of claim 8, wherein the request indicates a criteria, and the hardware processor is further configured to:
identify a candidate second entity that satisfies with the criteria;
identify at least one data type mapped to the candidate second entity;
train a model based on the at least one data type, wherein the model indicates a probability of usage of each data type among the at least one data type by the candidate second entity;
send the probabilities of usage of each data type to a device of the first entity; and
receive an indication that the candidate second entity is selected as the second entity to analyze the condition of the first entity.

14. A computer program product of preloading data for an analysis, the computer program product comprising a computer readable storage medium having program instructions embodied therewith, the program instructions executable by a processing element of a first device to cause the first device to:
receive a request for an analysis of a condition of a first entity, wherein the request comprises at least an identifier of the first entity and an identifier of the condition;
identify an interactive device that is mapped to the identifier of the first entity;
identify a data type that is mapped to the identifier of the condition;
determine that the interactive device is configured to provide interactive data of the data type;
retrieve the interactive data from the interactive device;
compile condition data based on at least the identifier of the first entity, the identifier of the condition, and the interactive data; and
load the condition data to a device associated with a second entity, wherein the loaded condition data is an input to the analysis of the condition of the first entity, and the analysis is to be performed by the second entity.

15. The computer program product of claim 14, wherein the program instructions are further executable by the processing element of the first device to cause the first device to schedule, autonomously, an appointment with the second entity to analyze the condition of the first entity.

16. The computer program product of claim 14, wherein the program instructions are further executable by the processing element of the first device to cause the first device to:
receive a notification from the device indicating an insufficiency of the loaded condition data;
retrieve additional interactive data of the data type from the interactive device;
compile new condition data based on the additional interactive data; and
load the new condition data to the device.

17. The computer program product of claim 14, wherein the request indicates an identifier of the second entity, and the program instructions are further executable by the processing element of the first device to cause the first device to identify the interactive device and the data type based on the second entity.

18. The computer program product of claim 14, wherein the request indicates an attribute, the program instructions are further executable by the processing element of the first device to cause the first device to:
identify a candidate second entity with the attribute;
identify at least one data type mapped to the candidate second entity;
send an identifier of the candidate second entity and the at least one data type to a device of the first entity; and
receive a selection indicating that the candidate second entity is selected as the second entity to analyze the condition of the first entity.

19. The computer program product of claim 14, wherein the request indicates a criteria, the program instructions are further executable by the processing element of the first device to cause the first device to:
identify a candidate second entity that satisfies with the criteria;
identify at least one data type mapped to the candidate second entity;
train a model based on the at least one data type, wherein the model indicates a probability of usage of each data type among the at least one data type by the candidate second entity;
send the probabilities of usage of each data type to a device of the first entity; and
receive an indication that the candidate second entity is selected as the second entity to analyze the condition of the first entity.

20. The computer program product of claim 19, wherein the request includes an identifier of the second entity, and the program instructions are further executable by the processing element of the first device to cause the first device to retrieve the interactive data from the interactive device based on the probabilities of usage of the data type by the second entity.
